(19) United States
(12) Patent Application Publication
(10) Pub. No.: US 2013/0204051 A1
(43) Pub. Date: Aug. 8, 2013

(54) Method And Apparatus For Heat Restoration In A Pervaporation Process Concentrating Ethanol

(71) Applicant: Antti Pasanen, Espoo (FI)

(72) Inventor: Antti Pasanen, Espoo (FI)

(73) Assignee: STI Biofuels OY, Helsinki (FI)

(21) Appl. No.: 13/579,879

(22) PCT Filed: Feb. 15, 2011
(86) PCT No.: PCT/FI11/50143

§ 371 Date: Oct. 5, 2012

(30) Foreign Application Priority Data
Feb. 19, 2010 (FI) 20105164

(51) Int. Cl. C07C 29/76; B01D 61/36

(52) U.S. Cl. CPC C07C 29/76; B01D 61/362
USPC 568/913; 210/180

(57) ABSTRACT

The invention relates to a method and an apparatus for dewatering mixture of ethanol and water. The method for dewatering mixture of ethanol and water in a pervaporation process arranged to dewater ethanol, to the retentate, from water as enriched to the permeate. In the method at least one of the streams of the permeate that is about to be cooled, before the entry of the stream into a vaporizer (2) arranged to vaporize at least partly once processed ethanol-water mixture by the pervaporation process, or another permeate stream upstream of pervaporation unit, is taken (HEXI) in the cooling phase of said at least one of the streams of the permeate, so that said heat taken is at least partly directed as secondary energy into the use for heating of the mixture of the ethanol and the water that is entering into the pervaporation unit, which is arranged to dewater the mixture of the ethanol and water by pervaporation. The invention relates also to a heat exchanger, use of the heat exchanger in general, but specifically in a pervaporation process for de-watering of ethanol-water mixture. The invention also relates to pervaporation unit comprising such a heat exchanger, and also to utility unit for ethanol rectification using the heat exchanger.

### FIELD OF THE INVENTION

The invention relates to a method for separation of mixture of a retentate substance component and a permeate substance component, in particular respectively ethanol and water, in a pervaporation process as defined in the preamble of independent claim 1.

The invention also relates to an apparatus for pervaporation based separation of mixture of a retentate substance component and a permeate substance component, in particular respectively ethanol and water, as defined in the preamble of independent claim 11.

The invention relates to pervaporation separation in a process that comprises distillation or dewatering or dehydration of mixture of a retentate substance component and a permeate substance component, in particular respectively ethanol and water preferably, but not necessarily only, from an ethanol content by volume of about 80 to about 96% to a ethanol content by volume of about 99.7%, which is suitable to be used as a component in fuel containing for example 85% ethanol and 15% gasoline (e85).

### OBJECTIVE OF THE INVENTION

The object of the invention is to provide an energy efficient novel and inventive method and apparatus for dewatering mixture of ethanol and water in a pervaporation process. In accordance of said object, it is a further object of the invention to provide an apparatus that is arranged to restore heat in the pervaporation process of the mixture as secondary energy for heating purposes of the mixture at the pervaporation phase In a further accordance, it is an object of the invention to provide a heat exchanger arrangement for the purpose. In an even further accordance, it is an object of the invention to use such heat exchanger in a pervaporation process, and/or as a part of the apparatus

### SHORT DESCRIPTION OF THE INVENTION

The method according to the invention is characterized by the definitions of independent claim directed to the method in a pervaporation process thereof

Preferred embodiments of the method are defined in the dependent claims that depend on said method claim in same category.

The apparatus of the invention is correspondingly characterized by the definitions of an independent claim directed to the apparatus

Preferred embodiments of the apparatus are defined in the dependent claims that depend on said method claim in same category.

Embodiments of the invention are combinable in suitable part.

In the method according to the invention, a mixture of a retentate and a permeate substance components, i.e. ethanol and water respectively is introduced in to an industrial scale process utilizing pervaporation by the processing apparatus for separating particularly ethanol from water. The mixture to be processed is heated at least partly by a secondary heat to the process temperature of the pervaporation process to occur in a corresponding process location for separation of the ethanol and water from each other. The secondary heat is taken from a material flow at least one process location to be used for the purpose. The mixture to be processed can be made at the utility site to the suitable grade for the processing and/or brought from another utility to be stored temporarily in a container or an ensemble of suitable containers for containing the mixture to be processed.

The separation is achieved by pervaporation unit which separates particularly ethanol and water from the said mixture into a first mixture and a secondary mixture. The first mixture is comprising water (for example 80% water, as high grade content) and ethanol, (for example 20% ethanol, as low grade content). The second mixture comprises ethanol (for example 99.7% ethanol, high grade content) and water (i.e. approximately 0.3% water, as low grade content).

The first mixture of ethanol and water is fed into a vaporizer from a pervaporation unit. In the vaporizer said mixture of ethanol and water is vaporized. The second mixture is either fed into a further process location of a further pervaporation process for a further treatment in a pervaporation unit, or when the rectification of the ethanol is on a desired level, out of the process, to the product storage for example.

The vaporizer is embodied in an exemplary embodiment by a distillation column. The distillation column can be situated, within a conformity of a boiler, but is not necessarily, if comprising the infrastructure for the distillation process with the facility for suitable adjusting of the temperature and/or pressure of the distillation column. According to an embodiment the distillation column comprises at least one subsidiary feeding point for a stream of material to be separated by vaporization from another, i.e. distilled. A first stream of the first mixture in vapor form is fed from the vaporizer to a heat exchanger unit. In the heat exchanger unit the vaporized mixture of ethanol and water is arranged to release heat to the heat exchanger and/or condense from a vapor phase to a liquid phase, while so cooled down, to form a liquid phase of said first mixture.

The second mixture in a second stream from the pervaporation unit is fed from the membrane unit to a storage, or to a further membrane unit for further rectification of the ethanol.

A secondary heat transferring stream comprising the second mixture in a stream of liquid phase mixture of ethanol and water is fed from the heat exchanger unit to at least one pervaporation unit comprising membrane unit of the pervaporation process. Thus, the second mixture is so preheated by the secondary energy at the process location. In such a pervaporation unit, at the process location in the process temperature and pressure, said stream of liquid phase mixture of ethanol and water is divided into a first stream of the first mixture and a second stream of the second mixture. A stream of vaporized mixture of ethanol and water is fed from the membrane unit, to be stored as such or to be further processed. At least one of said first and second streams is directed through at least one heat exchanger in a plurality of heat exchangers for restoration of the heat energy, to be used for heating the stream of vaporized mixture of ethanol and water as a second mixture, at a process location downstream the vaporizer but upstream the pervaporation unit comprising the membrane unit. This way, in the pervaporation process according to a preferred embodiment of the invention no other major heating units than that of the vaporizer in the vaporization unit is needed.

Instead of feeding ethanol comprising mixture merely into the temporal container at the beginning of the process cycle, an ethanol comprising mixture can be introduce into the subsidiary feeding point, and so directly into the distillation column acting as the vaporizer in a certain sense in an embodiment. This option can be used especially, but is not necessarily limited only to, when the mass flow of ethanol-water mixture is small and/or when the temperature of the subsidiary inputted fluid flow is near the separation temperature of the mixture in the vaporizer in the prevailing conditions therein.

In a preferred embodiment the retentate is rich in ethanol and the permeate is rich in water. Although ethanol and water are embodied as retentate and permeate substance components, other substance components can be separated accordingly by choosing the suitable retentate-permeate substance component pairs. When read and understood examples on the embodiments of the invention a skilled man in the art knows that many chemicals can be also so separated as such from each other by a pervaporation process. However, skilled man in the art realizes from the examples that for taking into account different properties of the chemicals as retentate and permeate substance components modifications may be necessary to the shown examples as embodiments for the ethanol and water as respective retentate and permeate substance components.

Thus in the following ethanol as a retentate and water as a permeate substance components are used as exemplary substance components for the embodiments to describe pervaporation processes according to the invention.

In addition, the pervaporation unit may comprise in an embodiment a heat exchanger, and/or an inter-heat exchanger arranged to warm up the liquid phase feed of the mixture of ethanol and water before the entry of the stream into the pervaporation unit comprising the membrane unit. Inter-heat exchanger may comprise a different substance component as a working fluid for taking the heat at a process location and releasing the heat at another process location for the pervaporation unit entering mixture of ethanol and water. Each membrane may be heated separately to the process temperature. Heating can be made by an inter-heating arrangement by using intermediate fluid for transferring the heat from the permeate flow to the membrane, or according to a variant directly via the heat exchange surface to the membrane.

By using a heat exchanger unit downstream the vaporizer, the vaporized mixture of ethanol and water vaporized in the vaporization unit, the heat of the stream can be at least partly taken to be restored for the use in another process location, such as the inter-heat exchanger for its warm-up for the stream of a mixture going through said inter-heat exchanger, (before or) in the pervaporation unit.

In a preferable embodiment of the invention said stream of liquid phase mixture of ethanol and water is divided in the pervaporation unit comprising the membrane unit, in a pervaporation step into a vaporized permeate stream of vaporized mixture of ethanol and water and into a liquid retentate stream of dewatered mixture of ethanol and water. In this preferable embodiment of the invention a vaporized permeate stream of vaporized mixture of ethanol and water is fed from the membrane unit. In this preferable embodiment of the invention a retentate stream of dewatered mixture of ethanol and water is removed/directed away from the membrane unit.

In a preferred embodiment of the invention said stream of liquid phase mixture of ethanol and water is divided in the pervaporation unit comprising the membrane unit of the pervaporation process in a pervaporation step into a vaporized permeate stream of vaporized mixture of ethanol and water and into a retentate stream of dewatered mixture of ethanol and water. In this preferable embodiment of the invention a retentate stream of dewatered mixture of ethanol and water is removed from the membrane unit as a product, intermediate- or by-product. In this preferable embodiment of the invention a vaporized permeate stream of vaporized mixture of ethanol and water is fed from the membrane unit to a cooling unit and the vaporized permeate stream of vaporized mixture of ethanol and water is condensed in the cooling unit to a liquid permeate from the vaporized mixture of ethanol and water. In this embodiment the energy taken by the cooling, implemented by at least one heat exchanger in a plurality of heat exchangers, is transferred so that at least a part of the heat is directed to the process location downstream from the vaporizer to heat the stream at a process location upstream the pervaporation unit. In this preferable embodiment of the invention the condensed liquid permeate stream of vaporized mixture of ethanol and water is fed from the cooling unit into a vaporizer, i.e. a distilling unit. In this preferable embodiment of the invention the liquid permeate stream of vaporized mixture of ethanol and water is divided in the distilling unit in a distillation step into a stream of vaporized mixture of ethanol and water and into a stream of water.

A heat exchanger for de-watering process of ethanol-water mixture via pervaporation according to a preferred embodiment has in the heat exchanger unit:
first means for a primary circulation of fluid for receiving heat to be transferred,
second means for a secondary circulation of fluid for giving heat received from said primary circulation fluid via an heat exchanging surface,
wherein the first means comprise connection means to connect to a permeate flow and/or a distillation product flow as a primary flow, and the second means comprise connection means to connect to the pervaporation unit heating circuit for heating the membrane unit.

An ethanol-water mixture de-watering process via pervaporation according to a preferred embodiment utilizes the heat exchanger according to a preferred embodiment.

A pervaporation unit according to a preferred embodiment of the invention is comprising a heat exchanger according to an embodiment of the invention as integrated into the pervaporation unit.

A utility unit/module for ethanol rectification according to a preferred embodiment of the invention is using the heat exchanger according to an embodiment of the invention in the pervaporation process for de-watering of ethanol-water mixture.

In a preferred embodiment of the invention a membrane of the pervaporation unit is comprising a membrane comprising a zeolite material such as Zeolite NaA or polymer membrane as preferably used in the membrane unit. An advantage of zeolite membranes is their relatively high maximum operating temperature, which means also durability and/or dissolving ability into the material streams. The membrane unit may consist of several membranes, for example membrane tubes, in series. Retentate is arranged to flow over the all membranes in use for the process and permeate comes through the membranes, in the process. However, the embodiments are not limited only to any particular topology of the series and/or in parallel in suitable part arranged array of membranes in the membrane unit for feed of ethanol-water mixture.

### LIST OF FIGURES

In the following the invention will described in more detail via examples by referring to the figures of which

FIG. 1A Illustrates embodiments according to the invention,

FIG. 1B shows a flow sheet of a simplified illustrative example on a preferred embodiment of the invention,

FIG. 1C illustrates a flow chart of a pervaporation based de-watering method according to embodiments of the invention, and

FIG. 2 shows a flow sheet of a more practically oriented illustration on a preferred embodiment of the invention.

### DETAILED DESCRIPTION OF AN ENSEMBLE OF EMBODIMENTS OF THE INVENTION

The figures show examples of a method and an apparatus according to the invention in an illustrative manner, without any intention to limit the scope only to the illustrated examples. In the following, vaporization of the fluid to be processed can be gained by evaporation, boiling, or by a combination thereof. According to an embodiment of the invention a practical implementation of the vaporization can be made by a distillation column, for example. However, the example is mentioned without any intention to restrict the embodiments only to the mere mentioned embodiment. A skilled man in the art knows many ways to implement the vaporizer for the pervaporation process. The distillation column as such can be operated in an embodiment variant in a substantial under-pressure, i.e. pressure conditions, where the pressure is substantially lower than the ambient pressure. In a further variant of such an embodiment, the pressure can be later increased, but not necessarily, by a compressor. However, for the ethanol and water mixtures, the column can be in an overpressure so that the produced vapors of the column yield a temperature range of 115-120° C. at the heat exchanger location for heating such a fluid that is arranged to heat the pervaporation membrane.

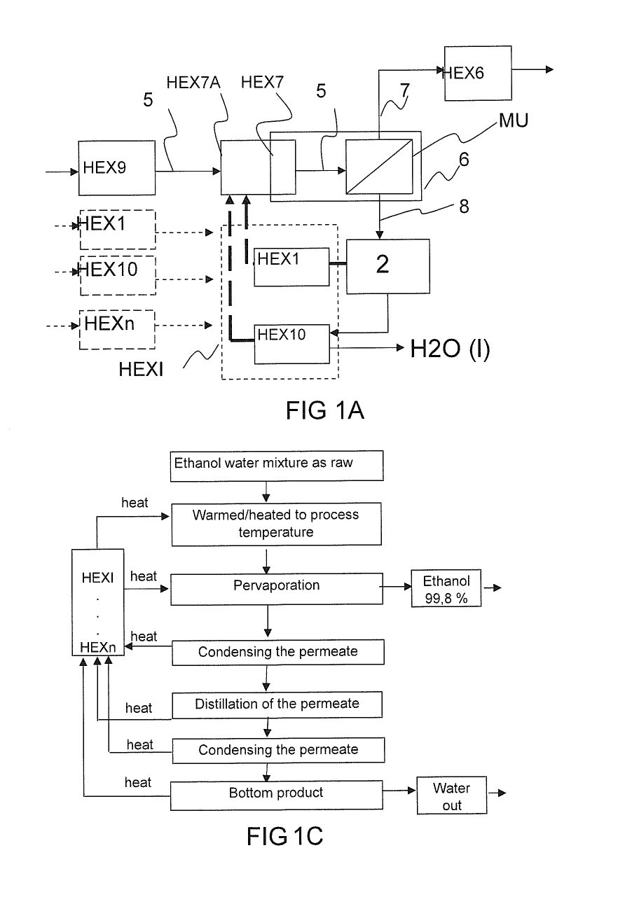

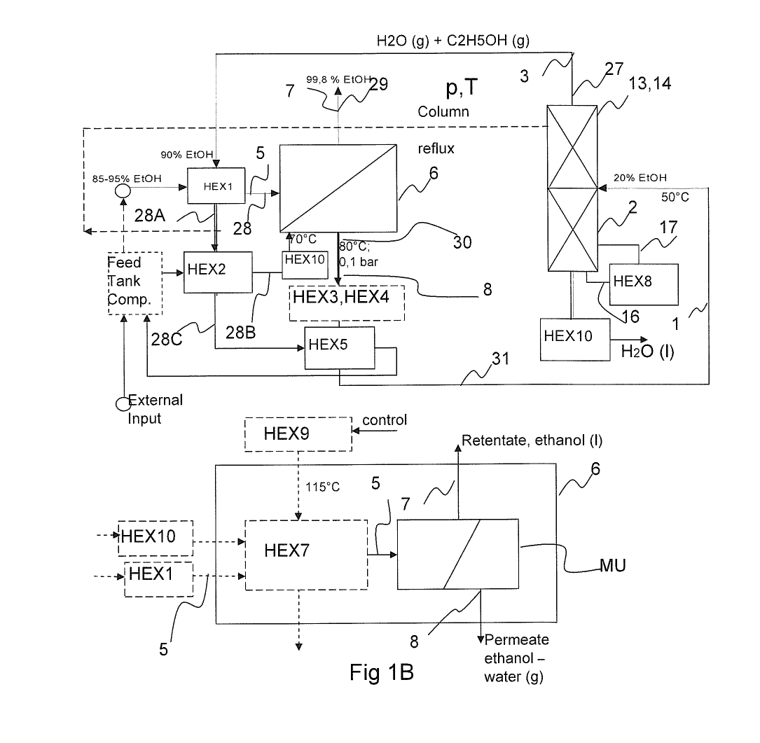

FIG. 1A shows an illustrative example on the process according to the invention in a very general level. The FIG. 1A show an ethanol rectification method by a pervaporation process, in which secondary energy is used for the heating of the membrane of the membrane unit MU arranged for de-watering of ethanol.

According to the embodiment shown in the illustration, ethanol is brought to the pervaporation unit 6 at the corresponding process location and conditions as such. The operation of the pervaporation unit is maintained as such in a normal way in typical conditions of pressure and temperature that a skilled man in the art knows very well for the membrane operation as such in an embodiment.

The ethanol mixture comprising water is brought in a liquid form to the pervaporation membrane, or an array of such. The array topology is not described in detail here although in some extent elsewhere. The array topology is neither limited for the embodiments. For simplicity reasons pervaporation membrane is used as an example, although the membrane topology as such were more complex than one or several membranes.

In the pervaporation process, the mixture of ethanol and water is fed in liquid form to the pervaporation unit membrane that lets the water go through so enriching the water in vapor form to the permeate at the opposite side of the membrane (than the entry side). The permeate is arranged to the flow 8 from the pervaporation unit for further processing by a distillation column 2. The feed line is illustrated by the reference number 8. The permeate is in vapor form, in the under pressure conditions, that are not further described, as a skilled man in the art knows how to maintain under pressure for a pervaporation process as such for a known membrane.

The mixture of ethanol and water in liquid form coming 5 to the membrane is flown over the membrane, and because of the under pressure (in magnitude about 100 mbar), the water mainly goes through the membrane, whereas the ethanol enriches to the retentate flowing 7 away from the pervaporation unit via the flow line illustrated by reference numeral 7. Heat can be taken from the flow 7 in the HEX6 before the mass flow of rectified ethanol product entry out of the process for storing or use otherwise. The ethanol content of the mixture of ethanol and water in the final product can be as high 99.7%, which is mentioned as an example without any intention to limit only to the mentioned content.

The permeate flow 8 is directed to the distillation column 2, so facilitating to restore the ethanol that have penetrated the membrane. So, the mixture of ethanol (20%) and water (80%) in the permeate flow is distilled, and the vaporized vapor form ethanol-water mixture is condensed and the heat is taken for use as heating energy by a heat exchanger (HEX1) for heating the pervaporation unit, and its membrane(s) to the process temperature at the process location where the pervaporation process is maintained. The heat so restored, as secondary energy, can be taken by a heat exchanger HEXI, which is provided by heat exchanger (HEX1) input and output for the mass flow from the distillation column 2 and the input and output for a mass flow that is arranged to heat the pervaporation unit.

In figure FIG. 1A, the HEXI is illustrated by a dashed line. Although in FIG. 1A there is HEX1 and HEX10 inside the dashed line disclosure as separate HEXes, the number of the HEXes is not limited to the shown example only for the permeate flow heat restoration, nor the topology of the HEXes. In FIG. 1A the HEX10 is arranged to take heat from the distillation column bottom discharge flow of the water to be discharged, and so to contribute to the HEXI's thermal circuit for transferring the heat to the pervaporation unit. Although the dashed thick lines from HEX1 and HEX10 are separated, to illustrate the heat transfer to the pervaporation unit, the flows can be embodied by an integrated fluid flow for the thermal energy transfer in the circuit. According to an embodiment the heat flows can be so arranged to branch that the conduits feed separate membranes and so maintain them in the process conditions in the pervaporation unit.

The pervaporation unit input is illustrated to occur via the HEX7 and/or HEX7A. As normally the pervaporation membrane unit is maintained in the pervaporation temperature by an inter heat exchanger utilizing a closed circuit flow for the heat transfer, the FIG. 1A illustrate that the heat from HEX1 is arranged to be transferred to the HEX7/HEX7A.

The way of drawing is just for illustrating various embodiments to deliver the heat from the HEXI to the pervaporation unit and consequently to the membrane unit via the HEX7/HEX7A. The HEX7 can be embodied as an internal inter-heat exchanger to the pervaporation unit (6), partly outside the pervaporation unit 6 as demonstrated by the HEX7/HEX7A or essentially an outsider inert-heat exchanger HEX7A. Any how, various combinations are possible embodiments to be applied in different kind of utilities for addressing the secondary energy restored by a HEXI heat exchanger in an appropriate topology of the individual process variant to the pervaporation unit and the membrane heating to the process temperature.

At the left hand side of the FIG. 1A, the HEX1, HEX9, HEX10, and HEXn are demonstrating that the ethanol-water mixture can enter via several mass flows to the pervaporation unit 6, so that the heat energy can be conserved and directed to the HEXI for utilization as secondary energy to restore heat as completely as possible to a certain extent of the main sources of the secondary heat from the process.

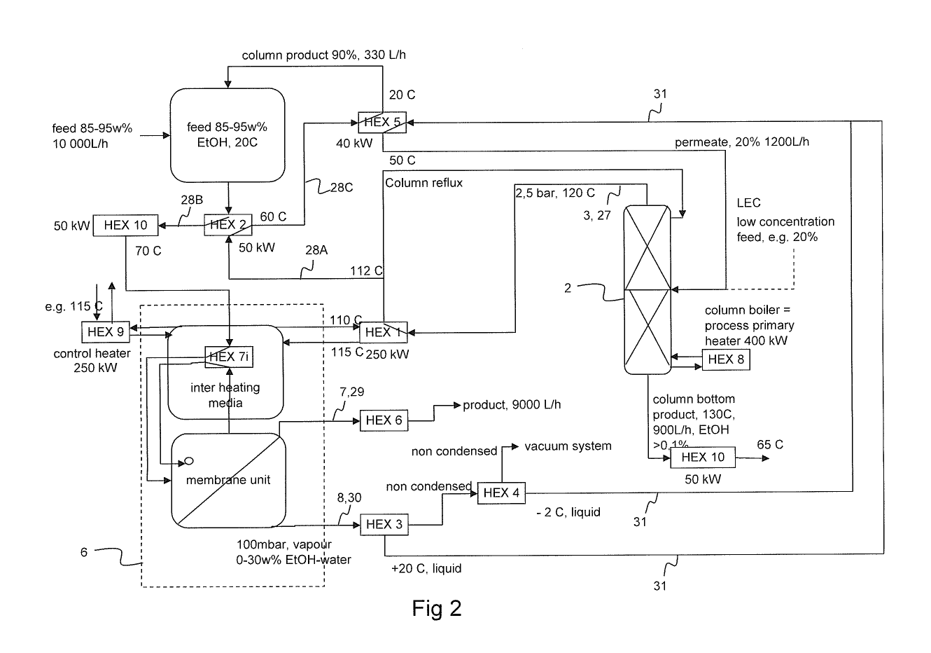

The illustrated apparatus shown in FIGS. 1B and 2 comprise a vaporizer 2 for receiving mixture of ethanol and water 1 and for vaporizing the mixture of ethanol and water 1. In an embodiment the vaporizer as such can be at least partly a distillation column 2 in the boiler that can comprise the infrastructure for maintaining the operations of the vaporizer 2 as a distillation column. Although the vaporizer can be implemented by a distillation column, but is not necessarily limited only such a device alone.

In the figures, in an exemplary embodiment the vaporizer 2 for heating and vaporizing mixture of ethanol and water comprising circulation means 13 and 14 for circulating vaporized mixture of ethanol and water inside the vaporizer 2 and finally through the vaporizer 2. In the figures, the column 2 can comprise a connection to a heat exchanging means HEX8 for transferring thermal energy from a fluid at locations 16 and 17 circulating via the heat exchanging means of the vaporizer 2 to the mixture of ethanol and water circulating through the vaporizer 2, so that the primary energy put into the HEX8 can be (directed) re-used for heating the mixture of ethanol and water in a process location elsewhere. The heat exchanger HEX10 can be used for taking the energy from the stream of water indicated by the symbols H₂O(1) the stream leading out of the boiler for the disposal. The HEX10 can be embodied as a separate unit from the boiler or as an integrated structure according to the respective embodiment variants, but with a connection to the column within the vaporizer 2 for the stream input from which the heat is about to be stored as secondary heat for heating use in a process location. In a preferred embodiment of the invention the pressure in a process location straight after the boiler product temperature is higher than inter-heating media temperature, resulting into column pressure 2.5 bar. (The HEX10 can be scaled to take heat energy so that the temperature decreases to half from the boiler bottom product temperature). In FIGS. 1 and 2 there are examples of preferable temperature and pressure ranges and examples of heat exchanger input and output temperatures, for ethanol-water mixtures. For other retentate substance component—permeate substance component pairs, the pressure can be higher than 200 mbar, but simultaneously equally or less than 10 bar. (The HEX10 can be scaled to take heat energy so that the temperature decreases to half from the boiler bottom product temperature). In FIGS. 1 and 2 there are examples of preferable temperature and pressure ranges and examples of heat exchanger input and output temperatures. In a preferred embodiment of the invention the pressure in a process location straight after the column 2 product temperature is higher than inter-heating media temperature, resulting into column pressure preferably about 2.5 bar

In the exemplary embodiments of the FIGS. 1B and 2, the reflux, for the distillation operation in the process utilizing the column 2 is indicated by a line from the HEX1 and/or HEX2.

The apparatus shown in FIG. 1B and 2 comprises also first conduit means 27 for feeding a stream of vaporized mixture of ethanol and water 3 from the vaporizer 2 to a heat exchange unit HEX1, which is arranged to be operable in the environmental conditions in respect of the temperature, pressure and material for heat exchanging. HEX1 is arranged for condensing totally or partly, the vapor of ethanol and water, even down to a temperature of condensation to liquid from said vaporized mixture of ethanol and water 3.

The embodied apparatus shown in FIG. 1B and/or FIG. 2 comprises also second conduit means 28 for feeding a stream of liquid phase mixture of ethanol and water 5 from the heat exchange unit HEX1 to a pervaporation unit 6 comprising a membrane unit for dividing said stream of liquid phase mixture of ethanol and water 5 into a stream in a vapor phase of mixture of ethanol and water 8 and into a stream of dewatered mixture of ethanol and water 7 i.e. a stream of mixture of ethanol and water 7 in liquid phase containing less water than said stream of liquid phase mixture of ethanol and water 5. The pervaporation unit as such may operate in a pressure higher than the ambient normal pressure i.e. in a range of 2.5 Bar to 2.5 Bar, but the permeate mixture of ethanol and water in vapor phase can be in a reduced pressure lower than the normal pressure. The vapor phase mixture of ethanol and water 3 is in over pressure (p) and in a higher temperature (T) than the inter-media heat exchanger, meaning preferably pressure range about 2.5 for column product vapor.

The pervaporation unit 6 comprising membrane unit, in an embodiment, consisting of one or several membranes, e.g. tubular membranes, in series, arranged to divide said stream of liquid phase mixture of ethanol and water 5 is preferably, but not necessarily, configured to divide said stream of liquid phase mixture of ethanol and water 5 in a pervaporation step into a vaporized permeate stream of vaporized mixture of ethanol and water 8 and into a retentate stream of dewatered mixture of ethanol and water 7.

The pervaporation unit 6 comprising membrane unit MU comprises preferably, but not necessarily, at least one of the following: a semi permeable membrane, a porous membrane, a ceramic membrane, a molecular sieve, and a membrane comprising a zeolite material such as zeolite NaA. The number and/or topology of the membrane units in the pervaporation unit as such are not limited to plain series connected, parallel connected and/or net-work of the series and parallel connected topologies alone.

The heat exchange unit HEX1 comprises preferably, but not necessarily, at least one of the following: a passive heat exchanger unit and a thermal pump. According to an embodiment a Joule-process as such can be utilized in a heat exchanger.

The apparatus shown in FIGS. 1B and 2 comprises third conduit means 29 feeding the stream of dewatered mixture of ethanol and water 7 from the pervaporation unit 6 comprising membrane unit MU.

If the pervaporation unit 6 comprising membrane unit for dividing said stream of pressurized mixture of ethanol and water 5 is configured to divide said stream of liquid phase mixture of ethanol and water 5 in a pervaporation step into a vaporized permeate stream of vaporized mixture of ethanol and water 8 and into a retentate stream of dewatered mixture of ethanol and water 7, the third conduit means 29 is configured for feeding a retentate stream of dewatered mixture of ethanol and water 7 from the pervaporation unit 6 comprising membrane unit MU, out of the process as the product. Conduits 28A, 28B and 28C indicate means for streaming ethanol-water mixture to the pervaporation unit and to HEX5, via the heat exchanger HEX2.

FIG. 1B also demonstrates the energy integration of internal parts of the membrane unit. In suitable part, the pervaporation unit 6 can also comprise a heat exchanger (HEX7) in some embodiments, in which the ethanol-water mixture entering pervaporation in the membrane unit MU and entering into each membranes is normally (commonly) heated in between the membranes. The heating of the retentate entering into the next membrane in membrane unit is heated by HEX7. HEX7 is getting the heating energy from HEX1 and HEX9. The heat integration of HEX1 and HEX9 with HEX7 via inter-heating media is can be done particularly as done in this invention. By this way by controlling the heat in the stream of the HEX7 the pervaporation process can be controlled in respect of the temperature in the pervaporation unit. HEX9 act as an control heater if HEX1 is not transferring enough energy to inter-heating media.

FIG. 1C illustrates a flow chart of a pervaporation based de-watering method according to embodiments of the invention. Accordingly, ethanol and water mixture is introduced to the process in liquid form. The material flow is heated to the process temperature. Secondary heat originating to the material flows of permeate of the process taken and restored in heat exchangers (HEXI, HEXn) is directed to the pervaporation unit for heating the membranes operated in the pervaporation process for dewatering the ethanol mixture as raw. The retentate maintaining in a liquid form is brought out of the process (ethanol 99.8 w %) as high grade rectified ethanol, comprising some fractioal water. The permeate, penetrating the membrane into a vapor phase, is cooled and in a variant of an embodiment the heat is taken and directed to the pervaporation unit/membrane heating via a heat exchanger. The permeate still comprising some ethanol (for example 20 w %) is vaporized in a vaporizer, which can be embodied by a distillation unit. The vaporized permeate can be condensed and the heat taken to be directed to the heat exchanger (HEXI, HEXn) to give the heat of the mass flow to the heat exchanger to be used in the pervaporation process temperature maintenance. According to an embodiment, the heat of the bottom product of the distillation unit can be taken and directed to a heat exchanger for the restoration o f the heat as secondary energy utilizable for heating the pervaporation unit and the related membranes, and/or the raw mixture entering to the process. Taking the heat refers to only a partial heat taking from the appropriate material flow, as the heat as such always preserves in some extent in the temperatures above the absolute zero in Kelvin. Thus, taking the heat refers only to partial taking of the heat as a skilled man in the art understands. The heat exchanging can be implemented by using a first fluid as a primary circuit fluid containing the heat to be given circulated in the primary circuit while maintaining the flow, and a secondary fluid circuit containing the heat receiving second fluid to be used directly or indirectly to heat the pervaporation unit and/or its membranes, or the raw ethanol flow entering the pervaporation unit. Between the primary circuit and secondary circuit there is the heat exchanging surface, adapted for the flow and the pressure as well as for the temperature prevailing in the circuit in question.

The apparatus shown in FIG. 2 comprises a pervaporation unit indicated by the numeral 6. According to an embodiment of the invention, the pervaporation unit 6 comprises the membrane unit further comprising the membrane that is arranged for the pervaporation based separation of the ethanol and water, to yield a first stream of permeate with a Low Ethanol Concentration water mixture (LEC) and a second stream of retentate stream with a High Ethanol Concentration water mixture (HEC). The ethanol content in the LEC can be up to 20% and in the HEC 99.8%, the residue comprising mainly water and in minor part other process specific impurities.

A conduit means 30 for feeding a vaporized permeate stream of vaporized mixture of ethanol and water 8 from the membrane unit 6 to a cooling unit HEX3 for condensing the vaporized permeate stream of vaporized mixture of ethanol and water 8 to a liquid permeate vaporized mixture of ethanol and water 10.

In FIG. 2 the cooling unit HEX3 can be provided with a circulation system for circulating a cooling fluids through the cooling unit. As said cooling fluids may a fluid flow in the apparatus having a lower temperature than said vaporized permeate stream of vaporized mixture of ethanol and water 8 be used. The FIG. 2 illustrates also another cooling unit HEX4, which can be operable for condensing a non-condensed component of the vapors from HEX3 into a liquid phase. Although the vacuum system is indicated to be connected to the conduit 30 via the HEX3 and HEX4 as embodied in the figure, a skilled person in the art knows from the embodiments, that the arrangement by HEX3 and HEX4 can be implemented in several ways, but not leaving the scope of the embodiments.

The apparatus shown in FIG. 2 comprises a conduit means 31 feeding a liquid permeate stream of vaporized mixture of ethanol and water 10 from the cooling unit HEX3 into a distilling unit 2 for dividing the liquid permeate stream of vaporized mixture of ethanol and water 8 in a distillation step into a stream of vaporized mixture of ethanol and water 12 into a stream of vaporized ethanol and water 27. The distilling unit 2, but not necessarily, comprises a distillation column as such. In the FIG. 2 the feed is arranged via streams 9 and/or 10 in corresponding conduits. According to an embodiment, a further low concentration feed can be brought to the distillation unit 2 via a branch joining to the conduit 31 thought the HEX5, or from a special line to the distillation unit 2.

In FIG. 2, according to an embodiment, the heat exchanger HEX5 is arranged to preheat the permeate from pervaporation unit and simultaneously tame the vaporized product from the distillation unit down to the storing temperature ethanol-water mixture in a temporal feeding storage, for storing the produced 80 w % ethanol in a feeding tank acting as a intermediate storage before the entry of the ethanol water mixture in a liquid phase into the pervaporation unit 6. The temperature in an embodiment may be for example approximately 20° C., but is not limited only thereto. As can be demonstrated via the FIG. 2 the condensation is not necessarily gained from vapor to liquid phase in one heat exchanger unit alone, rather than in several steps, of which at least certain ones are arranged for heat energy restoration for heating and/or warming a fluid flow in another process location. Thus the energy from the cooling can be taken and restore as heat in a process location where heating at the process location is expected.

In FIG. 2 according to an embodiment of the invention, HEX2 is arranged to cool down and/or tame the vaporized mixture of ethanol and water to liquid phase and simultaneously warm the feed to pervaporation unit to the temperature required for pervaporation.

Although in FIG. 2, the inputting of side streams into the distilling unit 2, is demonstrated by a symbolic feed line into the primary vaporizer, the vaporizer 2 can in an embodiment implemented in many ways as a skilled man in the art knows when read and understood the application.

The apparatus shown in FIGS. 1 and 2 comprises also preferably, but not necessarily, at least one flow means such as a pump for example a vacuum pump and/or a compressor for example a vacuum compressor for creating required flows through the various devices of the apparatus. Vacuum is sucked to permeate side, helping the permeate to diffuse over the membranes.

The method comprises a step for feeding mixture of ethanol and water 1 into a vaporizer 2. The method comprises a step for vaporizing said mixture of ethanol and water 1 in the vaporizer 2. The method comprises a step for pressurizing and raising the temperature of vaporized mixture of ethanol and water 3.

The method comprises a step for dividing said stream of mixture of ethanol and water 5 in the membrane unit 6 into a stream of mixture of ethanol and water 8 and feeding the stream of mixture of ethanol and water 8 from the membrane unit 6 and into a stream of dewatered mixture of ethanol and water 7 and feeding the stream of dewatered mixture of ethanol and water 7 from the membrane unit 6. This step of the method in preferably, but not necessarily, in the form of a step for dividing said stream of mixture of ethanol and water 5 in the membrane unit 6. In the method is preferably, but not necessarily, at least one of the following used in the membrane unit 6: semi permeable membrane, a porous membrane, a ceramic membrane, a membrane comprising a molecular sieve, and a membrane comprising a zeolite material. In an embodiment in which there are more than one membranes, at least some of them can be connected in series, i.e. so that the retentate flow of a first unit feeds the upstream unit of the series connected membranes in the membrane unit of the pervaporation unit. In an embodiment, some membranes are connected in parallel for parallel operation of the membrane topology. In a further embodiment, there are membranes that are arranged for parallel operation, but also membranes that are arranged in series for the serial operations in the same array. In one ensemble of embodiments a commercially available membrane unit as such is used.

In the method shown in FIG. 2 there is the vaporized permeate stream of vaporized mixture of ethanol and water 8 fed from the membrane unit 6 to a cooling unit HEX3, HEX4, and/or HEX5. In the method illustrated in FIG. 2, if the vaporized permeate stream of vaporized mixture of ethanol and water 8 in the cooling unit condensed to a liquid permeate vaporized mixture of ethanol and water and a liquid permeate stream of vaporized mixture of ethanol and water is fed into a distilling unit 2 that preferably, but not necessarily, comprises a conventional distillation column. In the method the latent heat of the stream from the cooling is directed to the pre-heating of the mixture of ethanol and water 5 entering to the pervaporation unit at a suitable process location.

In the method shown in FIG. 2 is the liquid permeate stream of mixture of ethanol and water in the distilling unit 2 divided in a distillation step into a stream of vaporized mixture of ethanol and water and into a stream of water, to be disposed via HEX10, that is arranged to restore the heat from the bottom product, apparent in the water coming from the column 2.

The inter-heating media can be arranged to warm at least one membrane unit of the pervaporation unit. The heating can be embodied in an embodiment so that for a series of operational pervaporation membranes, the inter-heating unit can heat the stream between the membranes the feed entering to the next pervaporation stage, i.e. the retentate of the previous stage downstream from said series connected pervaporation membranes.

In such embodiment, the heating energy can be brought to the inter-heat exchanger from the process locations apparent for the heat exchangers HEX1 and/or HEX9. The HEX7 is arranged to transfer the heat through the membranes flowing retentates. Combining this kind of utilization of inter heat exchanger to embodiments of the invention, an improved control can be gained for the combination over either use as alone. According to a variant of the embodiment, at least a part of the heat from the column can be brought to the HEX7. Thus, according to an embodiment the Hex7 may be configured fro receiving heat energy from several process locations so to better adapt to the condition of varying raw material grade to be processed and/or other conditions influencing to the heat balance of the process at the pervaporation unit.

The method comprises also preferably, but not necessarily, a step of pressing and/or sucking at least one of the streams of the method through at least one of the devices used in the method by means of at least one flow means such as a pump for example a vacuum pump and/or a compressor for example a vacuum compressor for creating required flows through the various devices of the apparatus, for generating, adjusting and/or maintaining the pressure conditions for the pervaporation conditions as such, i.e. for suitable under pressure at the side of the permeate penetrated a membrane in the pervaporation unit comprising a membrane topology.

Instead of feeding the ethanol comprising mixture into the container at the beginning of the process cycle, the mixture can be introduce into the subsidiary feeding point and into the vaporizer. In such a way distillation can be made, and various grades of ethanol can be rectified although the grade was not suitable as such for the pervaporation setup otherwise used. This can be made especially, when the mass flow is small and/or when the temperature of the subsidiary inputted fluid flow is near the n temperature of the mixture in the vaporizer in the prevailing conditions therein, so to have as little increase into the energy economy as possible in the circumstances applied.

It is apparent to a person skilled in the art that as technology advanced, the basic idea of the invention can be implemented in various ways. The invention and its embodiments are therefore not restricted to the above examples, but they may vary within the scope of the claims.

### EXAMPLE 1

An example on a practical implementation according to an embodiment is shown for embodying in a non-restrictive manner a process in accordance to the embodied invention, to restore the heat in the pervaporation process for ethanol concentration process from an ethanol-water mixture as secondary energy for further use in pre-heating. Reference is also made to the practical illustration on the example shown in FIGS. 1A, 1B and 2.

According to an embodiment of the invention, for the apparatus, known parts as such, commercially available as such, can be used in suitable extent in the process apparatus arranged to be used in the process. According to an embodiment of the invention a zeolite membrane is used in the example, but without any intention to restrict the scope of the example only to the mentioned membrane material.

Apparatus according to an embodiment of the invention at least in this example comprises at least one pervaporation unit which comprises at least one pervaporation dedicated membrane, or an ensemble of such comprising topologies of parallel and/or in series connected membranes. In series connected it is meant that one membrane, that is yielding the retentate so connected that the other membrane can use the said retentate as input feed. In parallel connected membranes such membranes utilize the same feed input. The membrane can be embodied as it was a member in a plurality of pervaporation membranes in a membrane unit comprising at least said membrane in the plurality arranged to have the membrane topology. Said at least one pervaporation membrane is arranged in the corresponding membrane unit so that the fluid, flown over the pervaporation membranes of the topology, is concentrated (for example to 99.8% of ethanol, in to the liquid phase as retentate) at the processing made by the membrane, and the membrane penetrated fluid, the permeate, after the membranes, in a vapor phase, comprises water approximately 80%, when observed from the corresponding flows after the pervaporation unit. The apparatus comprises also a vaporizer, such as a distillation column for purifying the separated 80% water from the residual ethanol that will be so concentrated from the 20% ethanol content back to the pervaporation membrane applicable feeding concentration. According to an embodiment of the invention the vaporizer is the same component in the same vaporizer 2 that has a subsidiary feed point for vaporizing the ethanol from a subsidiary input as raw-material feed of the mixture comprising ethanol and water, but the embodiment is not necessary so limited in respect of the vaporizer.

In the pervaporation process according to an embodiment the ethanol and water mixture feed is directed over the membrane as a fluid to form the product as in a liquid form, but the separable permeate component penetrates the membrane to form the permeate at the corresponding side of the membrane into a vapor form. According to an embodiment of the invention the raw ethanol-water mixture to be rectified advantageously comprises ethanol over 50 w %, preferably 80 w %-95 w % ethanol in the mixture, to form rectified ethanol.

Thus, there are advantages of the embodied pervaporation process as lower energy consumption and thus a higher efficiency than in another different conventional distillation process. The energy restoration from a permeate flow to heating the pervaporation process location can be made in a lower temperature than a mere distillation as such, and/or the membranes last longer in use in the process that is simple.

According to an embodiment of the invention, the pervaporation process made permeate as the first mixture is processed by a vaporizer, embodied by a distillation column, which is heated alone or optionally only in majorly by a primary heat source, i.e. a device arranged to release the energy fed external to the pervaporation process. This is possible, as there is at least one heat exchanger (HEXI) in a plurality of such heat exchangers that is arranged to utilize the process's internal energy available as secondary energy. This energy were otherwise released during the process if not restored via at least one said heat exchanger. Said secondary energy is thus directed to full fill the energy need of the pervaporation unit warming to the process temperature, entirely, or optionally at least in part.

The energy efficiency of the process is thus high as the low-ethanol-concentration (LEC)-flow, that penetrates the membrane, comprises energy that is used for heating or warming the feeding flow of the pervaporation unit. The implementation can be made by a pre-heat exchanger and giving the heat with HEXI for heating retentate flow with HEX7 in between the membranes in series. In commercial pervaporation units heating of membrane feed of each membrane in series is performed via inter-heating media and with HEX7. The heat integration with help of HEX1 and HEX9 is the particular heat integration of this example of embodied invention. The heat exchanger HEX1 and HEX9 can be embodied as a single one of either type, or as a combination of the mentioned types comprising an ensemble of members of each types, passive or active heat exchangers, active referring to heat-pump action for pumping heat from a medium in one temperature to another. Skilled man in the art knows several ways to combine the HEX1, HEX7 and HEX9 for configuring the heat energy flow with a medium from a process location upstream the pervaporation unit but downstream the distillation column.

An advantage of the embodiments is that they allow operating with a wide variety of feeding concentrations. Another advantage is that applicably the energy consumed in the process is relatively low, lower than in high-temperature based mere distillation processes. Thus, also flows with small thermal energy content can be utilized in various places of the process.

In the FIG. 1, the HEX1 is a heat exchanger (HEX) arranged to transfer heat energy of a vapor phase product to warm up a pervaporation membrane feed of each membrane via inter-heating media and HEX7. As a side effect, the vapor (coming from a distiller) that may be for example at a temperature of 120° C. so condenses totally or in part. HEX9 comprise means to control and tune that there is just enough heat energy in the pervaporation process. The HEX2 is arranged to cool down and condense the product (in vapor phase coming from the vaporizer) to a temperature in which the product turns to liquid and simultaneously the feed warms up at least near to the temperature required for feeding the pervaporation unit. HEX5 is arranged to preheat the permeate which is originating to the pervaporation unit, directly or via other HEX, HEX3 and/or HEX4. In the HEX5 the fluid flow coming from HEX2 simultaneously cools down to a temperature of approximately 20° C., the fluid vaporizer originating product which is comprising approximately 80 w % ethanol.

In an embodiment variant, into the vaporizer is fed raw-material mixture of ethanol and water in an inappropriately low concentrated ethanol mixture. This can be implemented by subsidiary input to bring such ethanol mixture into the process. The vaporizer is used to increase the ethanol concentration suitable level for the pervaporation unit. However, if the subsidiary input feed in an embodiment is used, this may affect to the primary energy consumption, if no secondary heat is used for warming according to an embodiment variant. In this way, the energy in the flow can be also utilized, and no additional primary energy source than the energy source for the vaporizer is needed

According to an embodiment of the invention illustrated in FIG. 2, the membrane unit directly or via the feeding flow is arranged to be heatable by heat exchanger HEX7. In the figure, the HEX7 is demonstrated to be an inter heating media-utilizing heat exchanger. According to an embodiment, HEX7 is configured to give heat directly, or via a working fluid indirectly, to the mixture entering a membrane unit in the pervaporation unit. The heat energy can be taken via the HEX9 that is configured to be a control heater arranged to control the heat. The heat can be taken from a suitable process location via exchanging the heat of a medium in the flow. The heat is then directed in a flow through the HEX7 utilizing the heat for heating at least one of the membranes. Similar way, the HEX1 can be used to take the heat energy available from the flow coming distillation column, of vaporized mixture of ethanol and water, to be returned at least partly via the reflux to the column, but the other part to the HEX2, and further to the HEX5 and even further to fed into the temporary storage container for raw ethanol. The column product can be 90% grade of ethanol.

The HEX2 and HEX10 can be arranged to heat the raw-ethanol feed coming from the tank, however preferably before the entry of the raw ethanol flow to the process location of the pervaporation unit, or before the HEX7 when used in the embodiment for pre-heating the mixture flow of ethanol and water entering the pervaporation unit.

### EXAMPLE 2

An optional process is disclosed in accordance of an example 1, but applied for a retentate substance component instead of ethanol so that the desired retentate is one of the following:

Methanol, Ethanol, Propanol (either of the isomers), Butanol (all isomers), Pentanol (all isomers), Cyclohexanol Benzyl alcohol (examples on alcohols); Benzene, Toluene, Phenol (examples on Aromatics); Methyl acetate, Ethyl acetate, Butyl acetate (examples on Esters) Acetic acid (example on an Organic Acid); Acetone Butanone Methyl isobutyl ketone (MIBK) (examples on Ketones); Triethylamine, Pyridine, Aniline, (examples on Amines), Methyl tert-butyl ether (MTBE), Ethyl tert-butyl ether (ETBE), Di-isopropyl ether (DIPE) Tetrahydro furan (THF) Dioxane(examples on Ethers); Chlorinated hydrocarbons (various), Dichloro methane, Perchloroethylene (examples on Aliphatics). A skilled person in the art of pervaporation knows that although the examples are shown in view point as the mentioned substance components were as retentate in a mixture, at lease some of the same substance components can be concentrated as permeates in suitable mixture of retentate and mixture. When read and understood the application text, a skilled man in the art can also consider the role of the individual substance components in a substance component pair of retentate and permeate selected from the above mentioned list of chemicals and water, so that would the substance component be a retentate or a permeate.

1. A method for dewatering mixture of ethanol and water comprising a pervaporation process arranged to dewater ethanol, to the retentate, from water as enriching to the permeate, wherein the heat, of at least one of the streams of the permeate that is about to be cooled, before the entry of the stream into a vaporizer arranged to vaporize at least partly once processed ethanol-water mixture by the pervaporation process, or another permeate stream upstream of pervaporation unit, is taken (HEXI) in the cooling phase of said at least one of the streams of the permeate, and said heat taken is at least partly directed as secondary energy into the use for heating of the mixture of the ethanol and the water that is entering into the pervaporation unit, which is arranged to dewater the mixture of the ethanol and water by pervaporation.

2. The method according to claim 1, comprising administering said secondary heat individually to each pervaporation membrane of said pervaporation unit.

3. The method according to claim 1, comprising steps for:
feeding mixture of ethanol and water into an vaporizer,
vaporizing said mixture of ethanol and water in the vaporizer and feeding a stream of vaporized mixture of ethanol and water from the vaporizer to a heat exchanger unit (HEX1) for condensing,
cooling down said mixture of ethanol and water to condense in the heat exchanger unit (HEX1) arranged co-operative with HEX7,
feeding a stream of liquid phase mixture of ethanol and water to each membrane of the membrane unit (MU) of the pervaporation process, and
dividing said stream of mixture of ethanol and water in the membrane unit (MU) in the pervaporation unit into a first stream of mixture of ethanol and water and feeding the stream of vaporized mixture of ethanol and water from the membrane unit (MU), and into a second stream of dewatered mixture of ethanol and water and feeding the stream of dewatered mixture of ethanol and water from the membrane unit (MU),
heating at a process location (6, HEX2, HEX7, HEX10) said entering stream of mixture of ethanol and water to the pervaporation process temperature by said secondary energy restored from at least one of the said first (HEX1, HEX3, HEX4, HEX5) and second (HEX6) streams directed through a heat exchanger (HEX1, HEX3, HEX4, HEX5, HEX6) that is arranged to transfer thermal energy from said at least one of the said first and second streams to at least one process location (6, HEX2, HEX7, HEX10) downstream the vaporizer but upstream the membrane of the membrane unit (MU) of the pervaporation process,

4. The method according to claim 1, wherein at least one of the following is used in the membrane unit (MU) of the pervaporation process: a semi permeable membrane, a porous membrane, a ceramic membrane, a membrane comprising a molecular sieve, and a membrane comprising a zeolite material.

5. The method according to claim 1, further including
dividing said stream of mixture of ethanol and water in the membrane unit of the pervaporation unit in a pervaporation step into a vaporized permeate stream of vaporized mixture of ethanol and water and feeding the vaporized permeate stream of vaporized mixture of ethanol and water from the membrane unit (MU) of the pervaporation unit and into a retentate stream of dewatered mixture of ethanol and water and feeding the retentate stream of dewatered mixture of ethanol and water from the membrane unit.

6. The method according to claim 5, further including
feeding the vaporized permeate stream of vaporized mixture of ethanol and water from the membrane unit (MU) to a cooling unit (HEX5) that is implemented by at least one heat exchanger in a plurality of heat exchangers (HEX5), to direct at least a part of the heat gained by the cooling to the process location (6, HEX2, (HEX7)) downstream from the vaporizer to heat the stream upstream the pervaporation unit,
condensing the vaporized permeate stream of vaporized mixture of ethanol and water in the cooling unit (HEX3, HEX4, HEX5) to a liquid permeate vaporized mixture of ethanol and water and feeding a liquid permeate stream of vaporized mixture of ethanol and water into a distilling unit, and
dividing the liquid permeate stream of vaporized mixture of ethanol and water in the distilling unit in a distillation step into a stream of vaporized mixture of ethanol and water and into a stream of water.

7. The method according to claim 6, wherein the process location to be heated comprises at least one membrane unit (MU) with one or several membranes in series in a pervaporation unit to be heated.

8. The method according to claim 7, wherein the process location to be heated is heated indirectly by means (HEX2, HEX10, HEX1, HEX9) and/or directly by means (HEX7) arranged to heat the stream via said means.

9. The method according to claim 1, wherein the process comprises feeding a stream of the column bottom product water to be disposed into a heat exchanger (HEX10) to be used as a source of heat energy for secondary energy for utilization in a process location for pervaporation (6, HEX7).

10. The method according to claim 1, wherein the process comprises feeding a stream of the high concentration ethanol into a heat exchanger (HEX1) for utilization as a source of heat energy for secondary energy.

11. An apparatus for dewatering mixture of ethanol and water in a pervaporation process wherein the apparatus, for dewatering mixture of ethanol and water in a pervaporation process is arranged for dewatering ethanol, to the retentate, from water to be enriched to the permeate, comprises a heat exchanger (HEXI) for exchanging heat energy from at least one of the streams of the permeate from the pervaporation unit to the stream of mixture of the ethanol and the water that is entering into the pervaporation unit for the dewatering of said mixture via pervaporation.

12. The apparatus according to claim 11 comprising:
a pervaporation unit for splitting a stream of mixture of ethanol and water into a water-rich permeate stream as a first flow and an ethanol rich retentate stream as a second flow,
a distillation unit for vaporizing the water rich permeate stream and for splitting the water-rich permeate stream into a water rich bottom discharge stream and an ethanol-rich top discharge stream, and
at least one heat exchanger (HEX1, HEX2, HEX5, HEX10) arranged so that at least one of the said first and second stream is directed through a heat exchanger (HEX1, HEX2, HEX5, HEX10,) for transferring thermal energy as secondary energy from said at least one of the said first and second streams to at least one process location (6, HEX7) downstream the vaporizer but upstream the membrane unit (MU) of the pervaporation process.

13. The apparatus according to claim 11, further comprising:
a vaporizer for receiving a mixture of ethanol and water and for vaporizing mixture of ethanol and water,
first conduit means for feeding a stream of vaporized mixture of ethanol and water from the vaporizer to a heat exchanger unit (HEX1) for condensing said vaporized mixture to liquid phase, and
second conduit means for feeding a stream of the mixture of ethanol and water from the heat exchanger unit (HEX1) to a pervaporation unit for dividing said stream of said mixture of ethanol and water into a stream of vapor phase ethanol and water and into a stream of dewatered mixture of ethanol and water.

14. The apparatus according to claim 11, wherein the pervaporation unit comprises in at least one membrane unit with one or several membranes in series at least one of the following: one or series of a semi permeable membrane, a porous membrane, a ceramic membrane, a molecular sieve, a membrane comprising a zeolite material and another pervaporation material comprising material according to a known techniques.

15. The apparatus according to claim 11, wherein at least one heat exchanger is arranged to be operable as a two-phase device so that it comprises first means for a first phase arranged to condense of the mixture from vapor phase to liquid phase and a second means for a second phase to cool the fluid either at a process location where the vapor phase is dominantly present or the liquid phase is dominantly present.

16. The apparatus according to claim 11, wherein
the pervaporation unit is configured to divide said stream of liquid phase mixture of ethanol and water in a pervaporation step into a vaporized permeate stream of said mixture of ethanol and water and into a retentate stream of dewatered mixture of ethanol and water.

17. The apparatus according to claim 16, further including
fourth conduit means for feeding the vaporized permeate stream of vaporized mixture of ethanol and water from the membrane unit to a cooling unit (HEX5) for condensing and/or cooling the vaporized permeate stream of vaporized mixture of ethanol and water to a liquid permeate mixture of ethanol and water, and
fifth conduit means feeding a liquid permeate stream of vaporized mixture of ethanol and water from the cooling unit (HEX5) into a distilling unit for dividing the liquid permeate stream of vaporized mixture of ethanol and water in a distillation step into a stream of vaporized mixture of ethanol and water and into a stream of water.

18. The apparatus according to claim 11, wherein the process location comprises at least one membrane unit at the process location.

19. The apparatus according to claim 11, wherein the apparatus comprises a reflux line arranged for a reflux flow from a heat exchanger (HEX1, HEX2) location of the process to the vaporizer,

20. The apparatus according to claim 11, wherein the apparatus comprises a heat exchanger (HEX9) arranged to be co-operable with another heat exchanger (HEX7) to control the heating of the membrane unit.

21. A heat exchanger for dewatering process of ethanol-water mixture via pervaporation, the heat exchanger unit comprising:
first means for a primary circulation of fluid for receiving heat to be transferred,
second means for a secondary circulation of fluid for giving heat received from said primary circulation fluid via a heat exchanging surface,
wherein the first means comprise connection means to connect at least to a permeate flow and/or a distillation product flow as a primary flow, and the second means comprise connection means to connect to the pervaporation unit heating circuit for heating a membrane unit in the membrane unit topology.

22.-25. (canceled)

26. The heat exchanger according to claim 21, wherein the dewatering process is an ethanol rectification process for restoring secondary energy for membrane heating.

27. The heat exchanger according to claim 21, wherein the dewatering process is a pervaporation process in an ethanol rectification process for restoring heat and directing the heat as secondary energy to membrane heating.
